(19) United States
(12) Patent Application Publication
(10) Pub. No.: US 2018/0055360 A1
(43) Pub. Date: Mar. 1, 2018

(54) MEDICAL INSPECTION APPARATUS AND MEDICAL INSPECTION SYSTEM

(71) Applicant: Konica Minolta, Inc., Tokyo (JP)

(72) Inventor: Takashi SAKAI, Yokohama-shi (JP)

(21) Appl. No.: 15/677,758

(22) Filed: Aug. 15, 2017

(30) Foreign Application Priority Data
Aug. 25, 2016 (JP) 2016-164298

(51) Int. Cl. A61B 5/00; G06F 19/00

(52) U.S. Cl. CPC A61B 5/0013; G06F 19/321; A61B 5/7425; A61B 5/0033; A61B 5/7435

(57) ABSTRACT

A medical inspection apparatus which is capable of communicating with an external device storing at least one piece of subject information of a subject and at least one piece of subject information update time information indicating an update time of the subject information and which inspects the subject to generate inspection data, the medical inspection apparatus including: an acquisitor which acquires the last updated subject information and the subject information update time information corresponding to the last updated subject information at a timing of execution of a predetermined operation input; and a setter which sets the acquired subject information as the subject information of an inspection target subject when it is within a predetermined time from the acquired subject information update time information.

### CROSS-REFERENCE TO RELATED APPLICATIONS

The present U. S. patent application claims a priority under the Paris Convention of Japanese Patent Application No. 2016-164298 filed on Aug. 25, 2016, the entirety of which is incorporated herein by references.

### BACKGROUND

### Technological Field

The present invention relates to a medical inspection apparatus and a medical inspection system.

### Description of the Related Art

Conventionally, there is known an electronic medical record terminal that is provided in a medical facility such as a hospital and used to acquire an electronic medical record from an electronic medical record database for all patients and to display the medical record as a findings screen. There is also known an ultrasound diagnostic apparatus that generates ultrasound image data by transmitting/receiving ultrasound waves to/from a patient as a subject.

The electronic medical record terminal and the ultrasound diagnostic apparatus are installed in a same examination room, for example. During ultrasound diagnosis inspection of a patient, the doctor manually inputs, into the ultrasound diagnostic apparatus, subject information (caption data), that is, basic information of the patient among electronic medical records displayed on the electronic medical record terminal. This increases doctor's operation load.

To cope with this, there is known an ultrasound image diagnostic apparatus that requests subject information to the electronic medical record terminal and receives responded subject information from the electronic medical record terminal (refer to Japanese Patent Application Laid-Open Publication No. 2014-018440).

There may be cases, however, where the electronic medical record terminal erroneously continues displaying the electronic medical record of a previously inspected patient at the time of inspection of an inspection target patient. In this case, the ultrasound image diagnostic apparatus cannot acquire subject information of the inspection target patient because the apparatus receives subject information of the patient preceding the inspection target. Moreover, there are not many electronic medical record terminals capable of responding with subject information in response to a request for the subject information.

### SUMMARY

An object of the present invention is to acquire subject information of an inspection target subject (patient) easily.

To achieve at least one of the abovementioned objects, according to an aspect of the present invention, a medical inspection apparatus reflecting one aspect of the present invention which is capable of communicating with an external device storing at least one piece of subject information of a subject and at least one piece of subject information update time information indicating an update time of the subject information and which inspects the subject to generate inspection data, the medical inspection apparatus including: an acquisitor which acquires the last updated subject information and the subject information update time information corresponding to the last updated subject information at a timing of execution of a predetermined operation input; and a setter which sets the acquired subject information as the subject information of an inspection target subject when it is within a predetermined time from the acquired subject information update time information.

### BRIEF DESCRIPTION OF THE DRAWINGS

The advantages and features provided by one or more embodiments of the invention will become more fully understand from the detailed description given hereinbelow and the appended drawings which are given by way of illustration only, and thus are not intended as a definition of the limits of the present invention:

FIG. 1 is a block diagram illustrating a first medical inspection system according to an embodiment of the present invention;

FIG. 2 is a block diagram illustrating a functional configuration of an electronic medical record terminal;

FIG. 3 is a block diagram illustrating a functional configuration of a clinical information server;

FIG. 4 is a block diagram illustrating a functional configuration of an ultrasound diagnostic apparatus;

FIG. 5A is a block diagram illustrating a functional configuration of an electronic medical record terminal and an ultrasound diagnostic apparatus in first inspection processing;

FIG. 5B is a sequence diagram illustrating a specific example of operation related to the first inspection processing;

FIG. 6 is a flowchart illustrating the first inspection processing;

FIG. 7A is a block diagram illustrating a functional configuration of an electronic medical record terminal and an ultrasound diagnostic apparatus in first subject information monitor processing and second inspection processing;

FIG. 7B is a sequence diagram illustrating a first specific example of the operation related to the first subject information monitor processing and the second inspection processing;

FIG. 8 is a sequence diagram illustrating a second specific example of the operation related to the first subject information monitor processing and the second inspection processing;

FIG. 9A is a flowchart illustrating the first subject information monitor processing;

FIG. 9B is a flowchart illustrating the second inspection processing;

FIG. 10 is a block diagram illustrating a second medical inspection system;

FIG. 11A is a block diagram illustrating a functional configuration of a clinical information server and an ultrasound diagnostic apparatus in third inspection processing;

FIG. 11B is a sequence diagram illustrating a specific example of operation related to the third inspection processing;

FIG. 12 is a flowchart illustrating the third inspection processing;

FIG. 13A is a block diagram illustrating a functional configuration of an electronic medical record terminal and an ultrasound diagnostic apparatus in second subject information monitor processing, fourth inspection processing, subject information deletion processing, and subject information transmission processing;

FIG. 13B is a sequence diagram illustrating a specific example of operation related to the second subject information monitor processing, the fourth inspection processing, the subject information deletion processing, and the subject information transmission processing;

FIG. 14A is a flowchart illustrating the second subject information monitor processing;

FIG. 14B is a flowchart illustrating the fourth inspection processing;

FIG. 14C is a flowchart illustrating the subject information deletion processing; and

FIG. 14D is a flowchart illustrating the subject information transmission processing.

### DETAILED DESCRIPTION OF EMBODIMENTS

Hereinafter, one or more embodiments of the present invention will be described with reference to the drawings. However, the scope of the invention is not limited to the disclosed embodiments.

The embodiments of the present invention, first, second, and third modifications will be described sequentially with reference to the drawings. In the following description, components having the same functions and configurations are denoted by the same reference numerals, and description thereof will be omitted.

### Embodiments

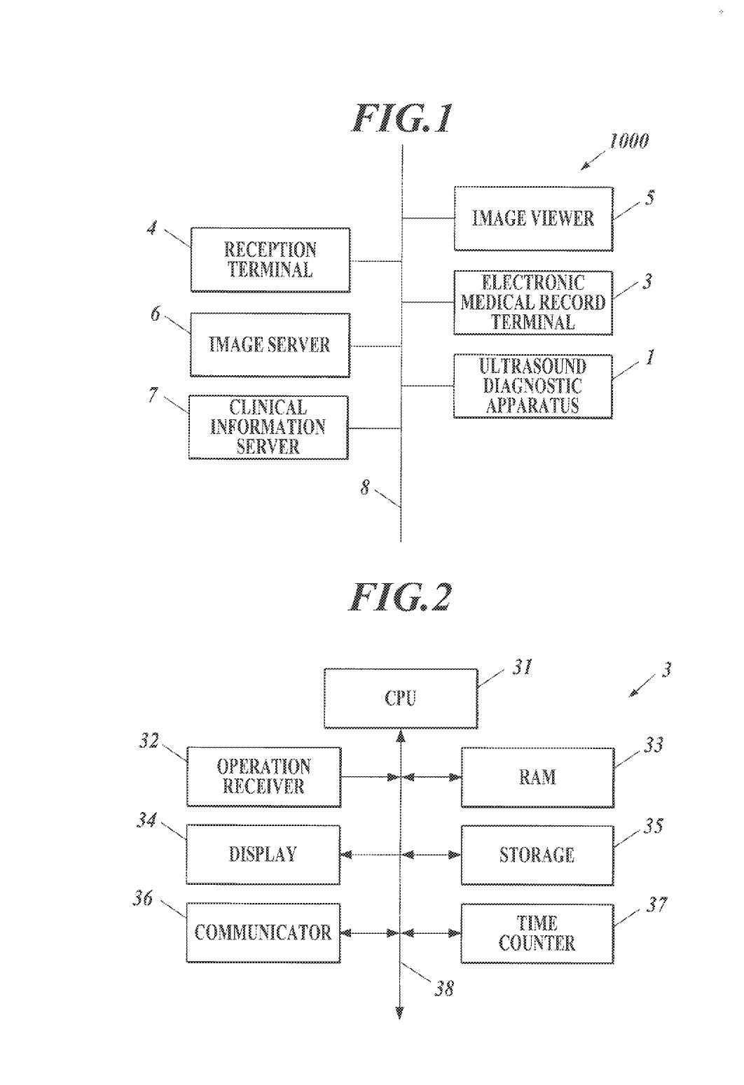

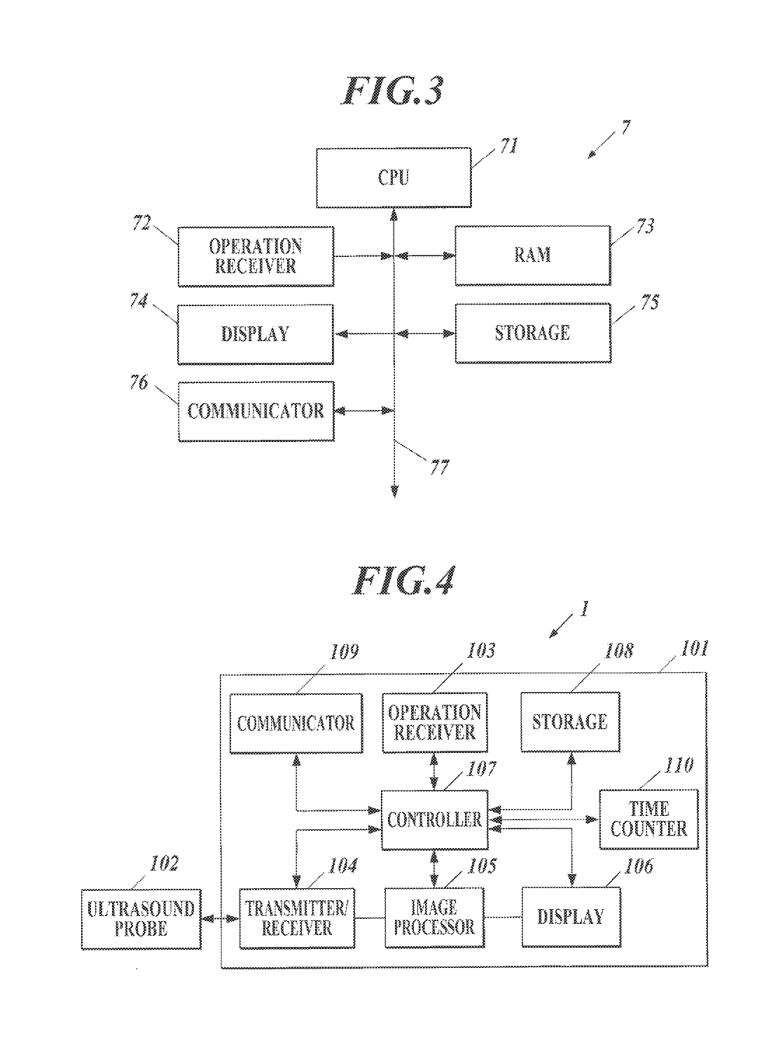

An embodiment according to the present invention will be described with reference to FIGS. 1 to 6. First, an apparatus configuration of the present embodiment will be described with reference to FIGS. 1 to 4. FIG. 1 is a block diagram illustrating a medical inspection system 1000 according to the present embodiment. FIG. 2 is a block diagram illustrating a functional configuration of an electronic medical record terminal 3. FIG. 3 is a block diagram illustrating a functional configuration of a clinical information server 7. FIG. 4 is a block diagram illustrating a functional configuration of an ultrasound diagnostic apparatus 1.

The medical inspection system 1000 of the present embodiment is a computer system installed in a medical facility. Herein, while the medical inspection system 1000 will be described as being installed in a hospital, for example, the location is not limited to this.

As illustrated in FIG. 1, the medical inspection system 1000 includes the ultrasound diagnostic apparatus 1 as a medical inspection apparatus, the electronic medical record terminal 3 as an external device, a reception terminal 4, an image viewer 5, an image server 6, and the clinical information server 7. Each of the apparatuses and devices of the medical inspection system 1000 is connected via a local area network (LAN) 8, for example.

Typically, a digital imaging and communication in medicine (DICOM) standard (Storage Service) is used as an image communication system in a hospital. Note that the communication method applicable to the present embodiment is not limited to this.

For example, herein, a hospital includes an examination room, a waiting room and a reception desk. At the reception desk, the reception terminal 4 that performs insurance score calculation, accounting calculation, or the like, is installed, and a receptionist is arranged. The receptionist gives, in order of reception, each of patients a reception number tag with an reception number (for example, a serial number in the order of acceptance of the day) printed for distinguishing individual patients (hereinafter may be referred to as subjects). Moreover, the receptionist inputs reception number, patient name, patient ID, or the like, into the reception terminal 4 in association with each other with reference to an examination card possessed by the patient, for example. The reception terminal 4 requests the clinical information server 7 to transmit an electronic medical record as clinical record data containing detailed patient information corresponding to the input patient name and/or patient ID.

After the reception terminal 4 has received the electronic medical record from the clinical information server 7, the receptionist confirms the content of the electronic medical record on a display screen of the reception terminal 4 and instructs transmission of the electronic record from the reception terminal 4 to the electronic medical record terminal 3. In response to this, the reception terminal 4 transmits the electronic medical record of the visiting patient to the electronic medical record terminal 3. The electronic medical record terminal 3 is installed in an examination room in which a doctor performs examination, diagnosis, or the like, of a patient, and displays the electronic medical record of the patient.

Furthermore, after finishing medical examination of the patient, the receptionist also performs a task of inputting necessary items of information related to accounting and medical bills (accounting calculation and insurance points billing calculation) into the reception terminal 4 on the basis of the information described in the electronic medical record. The receptionist confirms a medical treatment fee displayed on the display screen of the reception terminal 4 after completion of the input of the information on the medical bills, receives the medical treatment fee from the patient, and issues a medical examination receipt and passes it to the patient.

For example, the electronic medical record terminal 3 and the image viewer 5 are arranged on a medical examination desk (not illustrated) in the examination room, and the image server 6, the clinical information server 7, and the ultrasound diagnostic apparatus 1 are installed at predetermined positions in the examination room. The electronic medical record terminal 3 stores subject information of a plurality of examination target patients in the order of reception of the electronic medical records from the reception terminal 4. This operation puts the patients in examination waiting states. During this time, the patients wait in the waiting room. After examination target patient updating operation (display of findings screen) is performed by the doctor, the electronic medical record terminal 3 displays the findings screen including the content of the electronic medical record of a next examination target patient. Then, the doctor allows the examination target patient to enter the examination room and starts examination. The doctor performs examination and inputs an examination report into the electronic medical record terminal 3. The doctor performs inspection using the ultrasound diagnostic apparatus 1 as necessary. The ultrasound diagnostic apparatus 1 stores ultrasound image data acquired as a result of the inspection, in the image server 6.

The image server 6 includes an image database (DB) and stores image data such as ultrasound image data.

Image data stored in the image server 6 are read by the image viewer 5, and the medical image is displayed on the display screen of the image viewer 5. The doctor provides explanation to the patient using the medical image displayed on the display screen of the image viewer 5 and inputs findings into the electronic medical record terminal 3. When the examination is finished and the doctor inputs an instruction to finish the examination into the electronic medical record terminal 3, the electronic medical record terminal 3 transmits the electronic medical record to the reception terminal 4. Then, the patient leaves the examination room and returns to the waiting room. The doctor performs the examination target patient updating operation as described above, allows the next patient to enter the examination room, and performs examination.

Next, a functional configuration of the electronic medical record terminal 3 will be described with reference to FIG. 2. The electronic medical record terminal 3 includes a central processing unit (CPU) 31 as a deleter and a responsor, an operation receiver 32, a random access unit (RAM) 33, a display 34, a storage 35, a communicator 36, and a time counter 37. The individual parts of the electronic medical record terminal 3 are connected with each other via a bus 38.

The CPU 31 reads various programs such as a system program and a processing program stored in the storage 35, develops the programs in the RAM 33, and controls operation of individual parts of the electronic medical record terminal 3 in accordance with the developed program.

The operation receiver 32 includes a keyboard having a cursor key, numeric input keys, various function keys, or the like, and a pointing device such as a mouse, and outputs a depression signal of a key depressed on a keyboard and an input signal of a position by a pointing device or the like, to the CPU 31, as an operation signal.

The RAM 33 is a volatile semiconductor memory, and forms a work area that temporarily stores various programs executable by the CPU 31 in various types of processing executed by the CPU 31, read from the storage 35, input or output data, parameters, or the like.

The display 34 includes a monitor such as a liquid crystal display (LCD) and a cathode ray tube (CRT), and displays various screens in accordance with an instruction of a display signal input from the CPU 31.

The storage 35 is constituted with a hard disk drive (HDD), a nonvolatile semiconductor memory, or the like, and stores a system program and various programs executed by the CPU 31. In particular, herein, a first medical record management program is stored in the storage 35.

The communicator 36 is constituted with a network interface, or the like, and transmits/receives data to/from an external device connected to the LAN 8.

The time counter 37 is a real time clock, clocks the current date and time, and outputs current date and time information, to the CPU 31.

Next, a functional configuration of the clinical information server 7 will be described with reference to FIG. 3. The clinical information server 7 includes a CPU 71, an operation receiver 72, a RAM 73, a display 74, a storage 75, and a communicator 76. The individual parts of the clinical information server 7 are connected with each other via a bus 77. The CPU 71, the operation receiver 72, the RAM 73, the display 74, the storage 75, and the communicator 76 have similar functions as the CPU 31, the operation receiver 32, the RAM 33, the display 34, the storage 35, and the communicator 36, respectively, of the electronic medical record terminal 3, and thus, description thereof will be omitted.

Note that each of the reception terminal 4, the image viewer 5, and the image server 6 has a similar functional configuration as that of the electronic medical record terminal 3, and thus, descriptions of other devices will be omitted.

Next, a functional configuration of the ultrasound diagnostic apparatus 1 will be described with reference to FIG. 4. The ultrasound diagnostic apparatus 1 is a medical inspection apparatus that displays and outputs an ultrasound image of a state of an internal biological tissue of a patient (subject). That is, the ultrasound diagnostic apparatus 1 transmits an ultrasound wave (transmission ultrasound waves) to the inside of the subject and receives a reflected wave (reflected ultrasound wave: echo) of the ultrasound wave reflected inside the subject. The ultrasound diagnostic apparatus 1 converts the received reflected ultrasound wave into an electric signal and generates ultrasound image data on the basis of the electric signal. The ultrasound diagnostic apparatus 1 displays an internal state of the subject as an ultrasound image on the basis of the generated ultrasound image data. Moreover, the ultrasound diagnostic apparatus 1 generates supplementary information related to the generated ultrasound image data on the basis of information such as subject information, that is, information related to the patient. The ultrasound diagnostic apparatus 1 can generate an image file containing DICOM image data that conform to the DICOM standard by attaching the supplementary information to the ultrasound image data.

As illustrated in FIG. 4, the ultrasound diagnostic apparatus 1 includes an ultrasound diagnostic apparatus main body 101 and an ultrasound probe 102. The ultrasound probe 102 transmits a transmission ultrasound wave to the inside of the subject and receives a reflected ultrasound wave from the inside of the subject. The ultrasound diagnostic apparatus main body 101 is connected with the ultrasound probe 102 via a cable and transmits a drive signal of an electric signal to the ultrasound probe 102, thereby causing the ultrasound probe 102 to transmit the transmission ultrasound wave to the inside of the subject. Moreover, the ultrasound diagnostic apparatus main body 101 receives a reception signal, that is, an electric signal generated by the ultrasound probe 102 in accordance with the reflected ultrasound wave from the inside of the subject received by the ultrasound probe 102, and generates ultrasound image data as described above.

The ultrasound diagnostic apparatus main body 101 includes, for example, an operation receiver 103, a transmitter/receiver 104, an image processor 105, a display 106, a controller 107, a storage 108, a communicator 109, and a time counter 110. The operation receiver 103 serves as a selector and an operation receiver. The controller 107 serves as an acquisitor, a setter, a monitor, a display controller, and an output controller.

The operation receiver 103 includes various switches, buttons, a trackball, a mouse, and a keyboard, for performing inputs of commands for instructing the start of inspection, data such as personal information of the subject, and various parameters for displaying the ultrasound image on the display 106, and outputs an operation signal to the controller 107.

The transmitter/receiver 104 is a circuit that supplies a drive signal as an electric signal to the ultrasound probe 102 to cause the ultrasound probe 102 to generate a transmission ultrasound wave in accordance with the control of the controller 107 and that receives a reception signal an electric signal from the ultrasound probe 102. The transmitter/receiver 104 drives a plurality of transducers arranged in the ultrasound probe 102 to generate a transmission ultrasound wave to perform scanning. The transmitter/receiver 104 can perform reception beam forming by first amplifying the reception signal with a predetermined amplification factor and performing A/D conversion on the reception signal, and thereafter performing phasing addition processing on the signal. That is, the transmitter/receiver 104 constitutes an imager that images an inspection target site of the subject.

The image processor 105 can generate B mode image data by performing envelope detection processing, logarithmic compression, or the like, on the reception signal that has undergone phasing addition processing, and performing luminance conversion by adjusting the dynamic range and the gain. That is, the B mode image data represent intensity of the reception signal in luminance. The image processor 105 generates ultrasound image data in units of frames from the B mode image data generated in this manner. The ultrasound image data in units of frames may be referred to as frame image data. The image processor 105 includes a large capacity memory capable of holding frame image data for 10 seconds, for example.

A display device such as an LCD or a CRT display is applicable as the display 106. The display 106 displays the ultrasound image based on the frame image data generated by the image processor 105, on the display screen.

The controller 107 includes, for example, a CPU, a read only memory (ROM), and a RAM, reads various processing programs such as a system program stored in the ROM, develops the programs in the RAM, and controls each part of the ultrasound diagnostic apparatus 1 in accordance with the developed program. The RAM forms a work area for temporarily storing various programs to be executed by the CPU and data related to these programs. The ROM is constituted with a nonvolatile memory such as a semiconductor, and stores a system program for the ultrasound diagnostic apparatus 1, various processing programs such as an initial setting program and an ultrasound diagnostic program executable on the system program, various data, or the like. These programs are stored in the form of computer-readable program codes, and the CPU sequentially executes operation according to the program code. In particular, herein, the first inspection program is stored in the ROM.

The storage 108 is constituted by a large-capacity recording medium such as an HDD and can store the ultrasound image data generated as described above. Note that the storage 108 stores, as ultrasound image data, still image data, that is, image data of a still image for one frame, and moving image data generated to enable moving image display of the still image data of several frames. Moreover, in the present embodiment, subject information to be described below is also stored in the storage 108. Note that, in addition to the above-described HDD, it is possible to provide a portable recording medium such as a compact disk-recordable (CD-R) and a data read-write device such as a CD-R drive for recording data onto the portable recording medium, and to constitute the storage 108 with these. Moreover, the storage 108 may be configured to be capable of storing an image file containing the DICOM image data generated as described above.

The communicator 109 includes a LAN connector and transmits/receives data to/from each of the devices/apparatuses connected via the LAN 8.

The time counter 110 is a real time clock, clocks current date and time, and outputs current date and time information to the controller 107.

In each part of the ultrasound diagnostic apparatus 1, a portion of or all of the functions of individual functional blocks can be realized as hardware circuits such as an integrated circuit. An exemplary integrated circuit is a large scale integration (LSI). The LSI may be referred to as an IC, a system LSI, a super LSI, or an ultra LSI depending on the degree of integration. Moreover, an integrated circuit formation technique is not limited to LSI, but integration may be realized by a dedicated circuit or a general-purpose processor, or may be realized by using a field programmable gate array (FPGA) and a reconfigurable processor in which connection and setting of circuit cells inside the LSI can be reconfigured. Moreover, the functions of a portion or all of the individual functional blocks may be executed by software. In this case, the software is stored in one or more storage media such as a ROM, an optical disk, a hard disk, or the like, and the software is executed by the arithmetic processor.

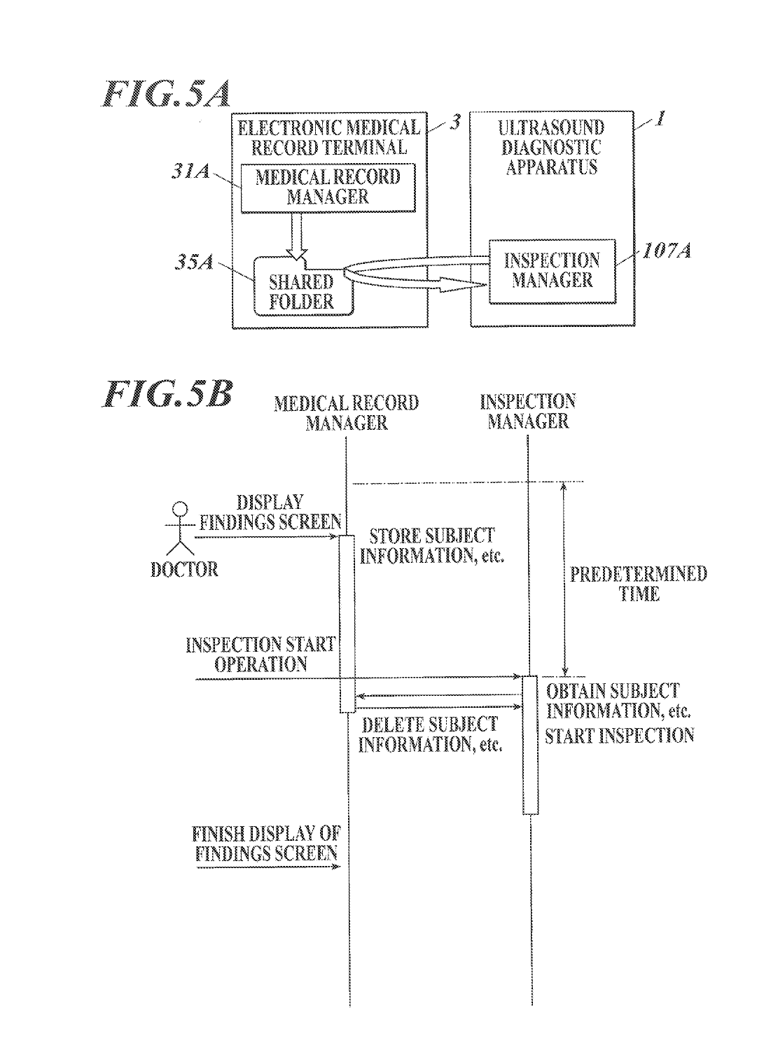

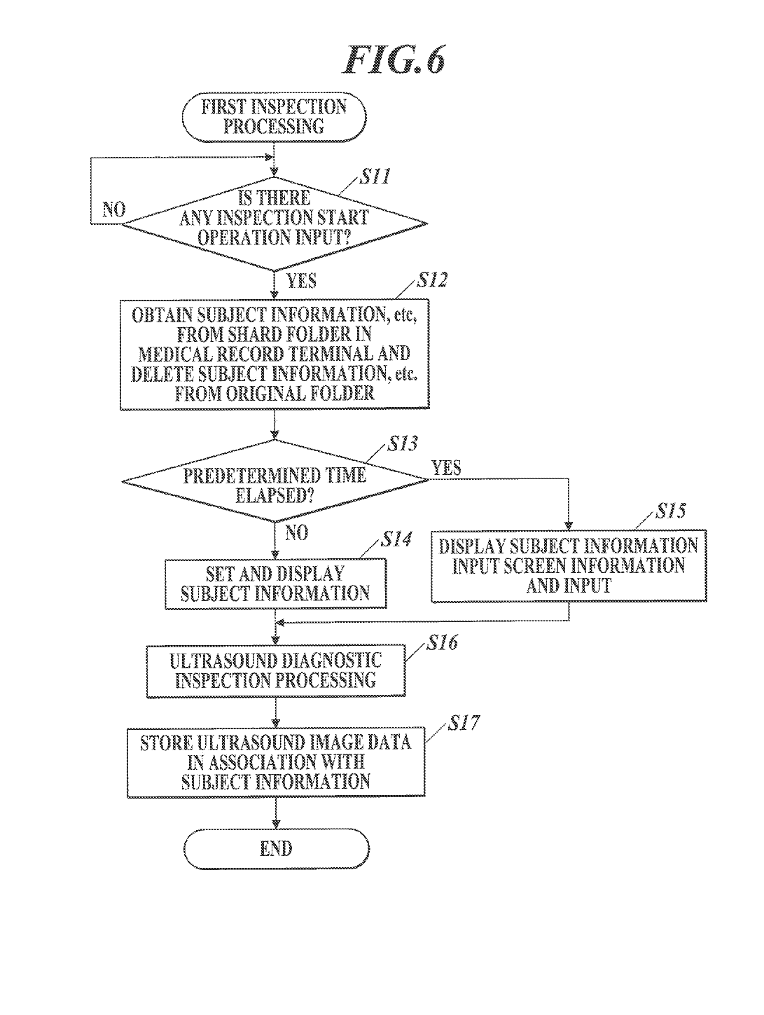

Next, operation of the medical inspection system 1000 will be described with reference to FIGS. 5 and 6. FIG. 5A is a block diagram illustrating functional configurations of the electronic medical record terminal 3 and the ultrasound diagnostic apparatus 1 in first inspection processing. FIG. 5B is a sequence diagram illustrating a specific example of operation related to the first inspection processing. FIG. 6 is a flowchart illustrating the first inspection processing.

As illustrated in FIG. 5A, in the electronic medical record terminal 3, the CPU 31 executes first medical record management processing in accordance with the first medical record management program stored in the storage 35. A medical record manager 31A is determined as an execution subject of the first medical record management processing. Moreover, the storage 35 stores subject information of an individual patient as a caption file of the electronic medical record, and also stores a shared folder 35A. Internal data of the shared folder 35A can be updated and deleted from the external ultrasound diagnostic apparatus 1. The subject information is basic information of the patient, and includes, for example, the patient's name, patient ID, and sex. The storage 35 also stores an electronic medical record folder (not illustrated) that stores an electronic medical record.

Upon receiving the electronic medical record of the visiting examination target patient from the reception terminal 4 via the communicator 36, the medical record manager 31A stores the electronic medical record in the electronic medical record folder. When the doctor inputs a findings screen display instruction for the next examination target patient via the operation receiver 32, the medical record manager 31A reads the electronic medical record of the next examination target patient from the electronic medical record folder, and creates findings screen information including the read electronic medical record, and displays the findings screen information on the display 34 as the findings screen. At the same time, the medical record manager 31A acquires current date and time information from the time counter 37, extracts subject information from the electronic medical record of the patient for which the findings screen information is being displayed, and stores the extracted subject information in the shared folder 35A in association with subject information update time information as the acquired current date and time information.

Then, triggered by an input of the first inspection processing execution instruction by the doctor via the operation receiver 103 in the ultrasound diagnostic apparatus 1, for example, the controller 107 executes the first inspection processing in accordance with the first inspection program stored in the ROM. An inspection manager 107A is determined as the execution subject of the first inspection processing.

As illustrated in FIG. 6, the inspection manager 107A initially determines whether ultrasound diagnosis inspection start operation input has been performed via the operation receiver 103 (Step S11). In a case where the inspection start operation input has not been performed (Step S11; NO), the processing proceeds to Step S11. In a case where the inspection start operation input has been performed (Step S11; YES), the inspection manager 107A acquires the subject information and the subject information update time information of the next examination target patient from the shared folder 35A of the electronic medical record terminal 3 via the communicator 109, and together with this, deletes the acquired subject information and subject information update time information from the shared folder 35A (Step S12).

Then, the inspection manager 107A acquires the current date and time information from the time counter 110, and determines whether a predetermined time fixedly set in advance has elapsed on the basis of the subject information update time information acquired in Step S12 using the acquired current date and time information (Step S13). The predetermined time in Step S13 is a passage of time for determining whether the findings screen of the already examined patient is erroneously left on, and an exemplary predetermined time is five minutes.

In a case where the predetermined time has not elapsed (Step S13; NO), the inspection manager 107A sets the subject information acquired in Step S12 as the subject information of the inspection target patient and displays the information on the display 106 (Step S14). In a case where the predetermined time has elapsed (Step S13; YES), the inspection manager 107A generates subject information input screen information for inputting the subject information and displays the generated information on the display 106, and receives an input of the subject information of the next inspection target patient from the doctor via the operation receiver 103 (Step S15).

Then, on the basis of various operation inputs from the doctor via the operation receiver 103, the inspection manager 107A performs ultrasound diagnosis inspection processing of the inspection target patient and generates ultrasound image data (Step S16). The doctor applies the ultrasound probe 102 to subject as the inspection target patient, generates ultrasound image data, and performs various operation inputs such as freeze operation.

On the basis of the input of an instruction to store the ultrasound image data from the doctor via the operation receiver 103, the inspection manager 107A stores storage target ultrasound image data generated in Step S16 in the image server 6 (Step S17) in association with the subject information acquired in Step S12 or input in S15, via the communicator 109, and finishes the first inspection processing.

Next, a specific example of the operation related to the first inspection processing will be described with reference to FIG. 5B. First, one examination target patient who has finished reception enters the examination room. Then, the doctor performs operation input of an instruction to display the findings screen including the electronic medical record of the examination target patient via the operation receiver 32, and the findings screen information is displayed on the display 34. In response to this, the medical record manager 31A of the electronic medical record terminal 3 stores the subject information of the examination target patient together with the subject information update time information, in the shared folder 35A.

Then, the doctor performs operation input of an ultrasound diagnosis inspection start instruction for the inspection target patient via the operation receiver 103 of the ultrasound diagnostic apparatus 1, and the inspection manager 107A acquires, in Step S12, the subject information of the examination target patient and the subject information update time information from the shared folder 35A, and deletes the acquired subject information and the subject information update time information in the shared folder 35A. Then, the inspection manager 107A determines, in Step S13, that the acquired subject information update time information has not passed a predetermined time from the current date and time information, and the inspection manager 107A sets, in Step S14, the acquired subject information as the subject information of the inspection target patient. Then, in Steps S16 and S17, the inspection manager 107A starts ultrasound diagnostic inspection and stores the ultrasound image data generated in the inspection in the image server 6 in association with the subject information of the inspection target patient. After the examination is finished, the doctor performs operation input of an instruction to finish the display of the findings screen of the examination target (inspection target) patient via the operation receiver 32, and then, the display of the findings screen information on the display 34 is finished.

As described above, according to the present embodiment, the ultrasound diagnostic apparatus 1 is capable of communicating with the electronic medical record terminal 3 that stores patient subject information and subject information update time information, inspects the patient, and generates ultrasound image data. The ultrasound diagnostic apparatus 1 includes the controller 107 (inspection manager 107A) that acquires the last updated subject information and subject information update time information at the timing of execution of a predetermined operation input, and sets the acquired subject information as the subject information of the inspection target patient in a case where the time is within a predetermined time from the acquired subject information update time information. With this configuration, it is possible to easily acquire and set the subject information of the inspection target patient.

Moreover, the predetermined operation input is an inspection start operation input. Therefore, it is possible to acquire subject information of the inspection target patient together with necessary operation, and thus to reduce the operation burden.

Moreover, the ultrasound diagnostic apparatus 1 acquires from the electronic medical record terminal 3 the last updated subject information and subject information update time information at a timing of execution of the predetermined operation input. With this configuration, the subject information is not accessed at a predetermined cycle, making it possible to reduce the frequency to access the subject information and to reduce the processing load of the ultrasound diagnostic apparatus 1.

Moreover, the ultrasound diagnostic apparatus 1 includes the ultrasound probe 102 that performs ultrasound diagnosis inspection on the patient (subject) and generates ultrasound image data, the transmitter/receiver 104, and the image processor 105. The inspection manager 107A outputs and stores the generated ultrasound image data onto the image server 6 in association with the set subject information. With this configuration, it is possible to easily associate the ultrasound image data with the subject information.

The ultrasound diagnostic apparatus 1 further includes the operation receiver 103 that receives an operation input of the subject information of the inspection target patient in a case where a predetermined time has elapsed from the acquired subject information update time information. With this configuration, it is possible to reliably acquire the subject information of the inspection target patient by operation input even in a case where the acquired subject information does not belong to the inspection target patient.

### First Modification

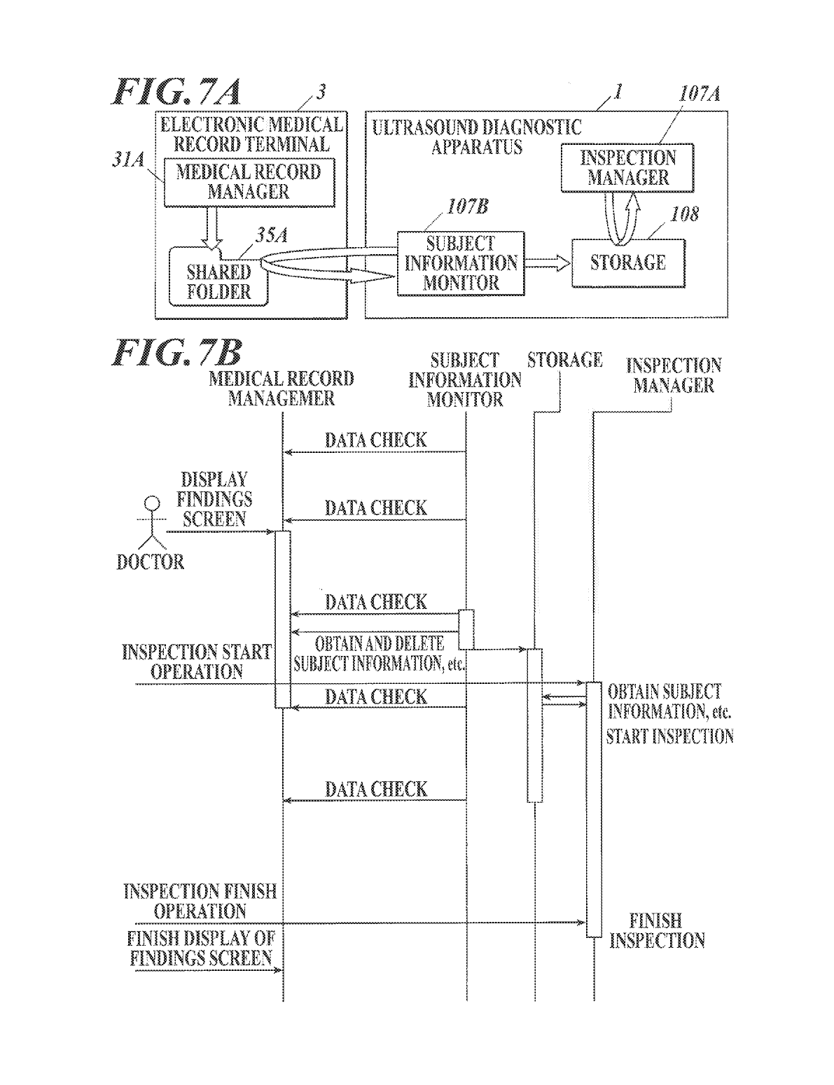

A first modification of the above-described embodiment will be described with reference to FIGS. 7A to 9. Similarly to the above-described embodiment, a medical inspection system 1000 is used as the apparatus configuration of the present modification. Note that the ROM of the controller 107 of the ultrasound diagnostic apparatus 1 stores a first subject information monitor program and a second inspection program instead of the first inspection program.

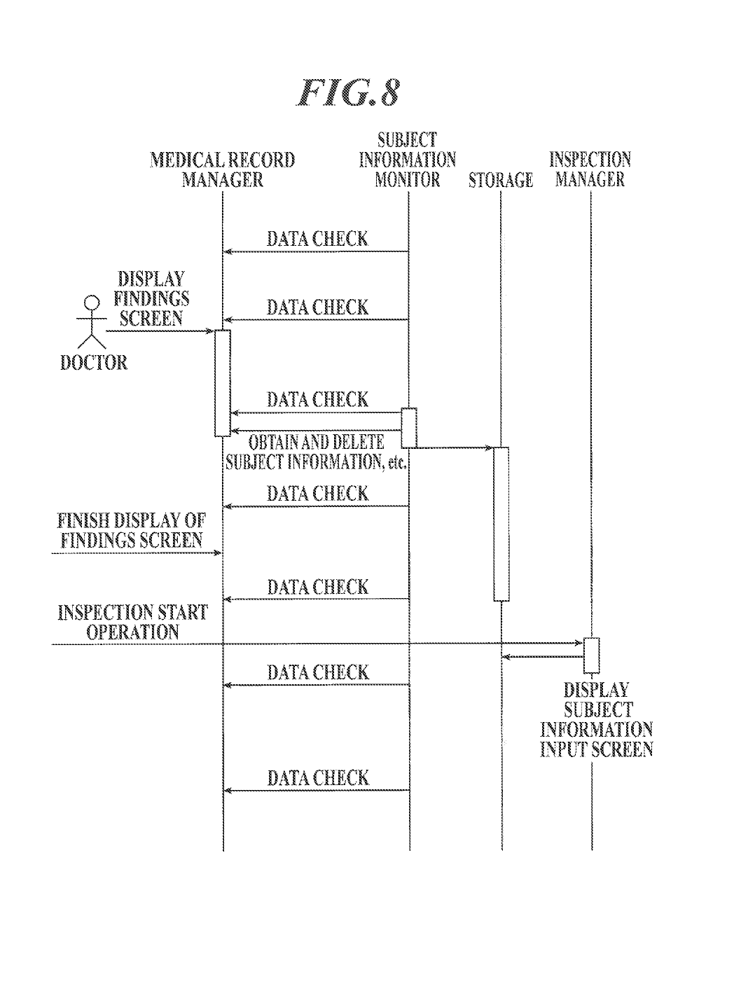

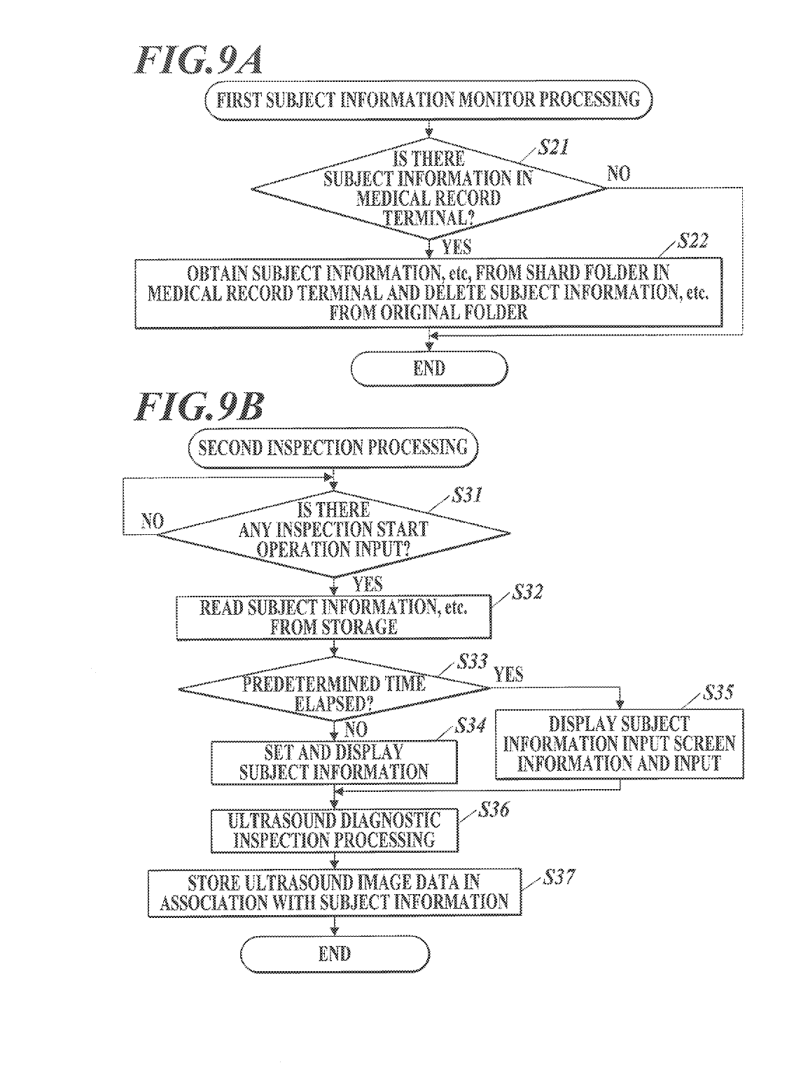

Next, operation of the medical inspection system 1000 will be described with reference to FIGS. 7A to 9. FIG. 7A is a block diagram illustrating functional configurations of the electronic medical record terminal 3 and the ultrasound diagnostic apparatus 1 in the first subject information monitor processing and second inspection processing. FIG. 7B is a sequence diagram illustrating a first specific example of the operation related to the first subject information monitor processing and the second inspection processing. FIG. 8 is a sequence diagram illustrating a second specific example of the operation related to the first subject information monitor processing and the second inspection processing. FIG. 9A is a flowchart illustrating the first subject information monitor processing. FIG. 9B is a flowchart illustrating the second inspection processing.

First, as illustrated in FIG. 7A, similarly to the above-described embodiment, the medical record manager 31A is determined as the execution subject of the first medical record management processing in the electronic medical record terminal 3, and accordingly, the medical record manager 31A stores electronic medical records in the electronic folders, generates the subject information and the subject information update time information of the examination target patient, and stores the generated information in the shared folder 35A in accordance with the operation input of the instruction to display the findings screen of the next examination target patient.

In parallel with this, the first subject information monitor processing is executed. The first subject information monitor processing is executed repeatedly at a predetermined cycle. In the ultrasound diagnostic apparatus 1, triggered by the passage of a predetermined cycle of the current date and time information of the time counter 110 from the previous execution of the first subject information monitor processing, the controller 107 executes the first subject information monitor processing in accordance with the first subject information monitor program stored in the ROM. A subject information monitor 107B is determined as the execution subject of the first subject information monitor processing.

As illustrated in FIG. 9A, the subject information monitor 107B first checks (polls) data in the shared folder 35A of the electronic medical record terminal 3 via the communicator 109, and then, determines whether the subject information is stored (Step S21). In a case where the subject information is not stored (Step S21; NO), the first subject information monitor processing is finished. In a case where the subject information is stored (Step S21; YES), the subject information monitor 107B acquires subject information and subject information update time information of the next examination target patient from the shared folder 35A via the communicator 109 and stores the acquired information in the storage 108, and together with this, deletes the acquired subject information and subject information update time information from the shared folder 35A (Step S22), and finishes the first subject information monitor processing.

Then, triggered by an input of the second inspection processing execution instruction by the doctor via the operation receiver 103 in the ultrasound diagnostic apparatus 1, for example, the controller 107 executes the second inspection processing in accordance with the second inspection program recorded in the ROM. The inspection manager 107A is determined as the execution subject of the second inspection processing.

As illustrated in FIG. 9B, Step S31 is similar to Step S11 of the first inspection processing in FIG. 6. In a case where the inspection start operation input is performed (Step S31; YES), the inspection manager 107A reads the subject information and the subject information update time information of the examination target patient stored in the Step S22, from the storage 108 (Step S32). Steps S33 to S37 are similar to Steps S13 to S17 in the first inspection processing in FIG. 6.

Next, a first specific example of the operation related to the first subject information monitor processing and the second inspection processing will be described with reference to FIG. 7B. First, one examination target patient who has finished reception enters the examination room. Then, the doctor performs operation input of an instruction to display the findings screen including the electronic medical record of the examination target patient via the operation receiver 32, and then, the findings screen information is displayed on the display 34. In response to this, the medical record manager 31A of the electronic medical record terminal 3 stores the subject information of the examination target patient together with the subject information update time information, in the shared folder 35A.

In parallel with this, the subject information monitor 107B repeatedly executes the first subject information monitor processing at a predetermined cycle. Subsequently, in Step S22, the subject information monitor 107B acquires the subject information and the subject information update time information of the examination target patient from the shared folder 35A, stores the acquired information in the storage 108, and deletes the acquired subject information and the subject information update time information in the shared folder 35A.

Then, the doctor performs operation input of an ultrasound diagnosis inspection start instruction for the inspection target patient via the operation receiver 103 of the ultrasound diagnostic apparatus 1, and the inspection manager 107A reads, in Step S32, the subject information and the subject information update time information of the patient from the storage 108. Then, the inspection manager 107A determines, in Step S33, that the read subject information update time information has not passed a predetermined time from the current date and time information, and sets, in Step S34, the read subject information as the subject information of the inspection target patient. In Steps S36 and S37, the inspection manager 107A starts ultrasound diagnostic inspection and stores the ultrasound image data generated in the inspection in the image server 6 in association with the subject information of the inspection target patient. Then, the doctor performs operation input of an instruction to finish the ultrasound diagnosis inspection via the operation receiver 103 of the ultrasound diagnostic apparatus 1, and the inspection of the ultrasound diagnostic apparatus 1 is finished. After the examination is finished, the doctor performs operation input of an instruction to finish the display of the findings screen of the examination target patient via the operation receiver 32, and then, the display of the findings screen information on the display 34 is finished.

Next, a second specific example of the operation related to the first subject information monitor processing and the second inspection processing will be described with reference to FIG. 8. Display of the findings screen information and the first subject information monitor processing by the subject information monitor 107B are similar to the first specific example of the present modification. Herein, for example, this is a case where the subject information stored in the shared folder 35A belongs to the previously examined patient and the findings screen information of the examined patient is erroneously left on. Accordingly, the doctor performs operation input of an instruction to finish the display of the findings screen of the examination target patient via the operation receiver 32, and then, the display of the findings screen information on the display 34 is finished.

Then, the doctor performs operation input of an ultrasound diagnosis inspection start instruction for the inspection target patient via the operation receiver 103 of the ultrasound diagnostic apparatus 1, and the inspection manager 107A reads, in Step S32, the subject information and the subject information update time information of the patient from the storage 108. Then, in Step S33, the inspection manager 107A determines that the read subject information update time information has passed a predetermined time from the current date and time information. This is because the read subject information belongs to the previously examined patient and a long time has elapsed since the start of display of the findings screen information.

Subsequently, in Step S35, the inspection manager 107A displays the subject information input screen information on the display 106, and receives an operation input of the subject information of the inspection target patient from the doctor via the operation receiver 103. The inspection manager 107A starts ultrasound diagnostic inspection and stores the ultrasound image data generated in the inspection in the image server 6 in association with the subject information of the inspection target patient.

As described above, according to the present modification, the ultrasound diagnostic apparatus 1 includes the storage 108 and the controller 107 (subject information monitor 107B) that checks, at a predetermined cycle, whether the electronic medical record terminal 3 stores the subject information, and in a case where the electronic medical record terminal 3 stores the subject information, the controller 107 acquires the subject information and the subject information update time information and stores the acquired information in the storage 108. The inspection manager 107A reads and acquires, from the storage 108, the last updated subject information and subject information update time information at a timing of execution of the predetermined operation input. With this configuration, it is possible to easily and reliably acquire and set the subject information of the inspection target patient.

### Second Modification

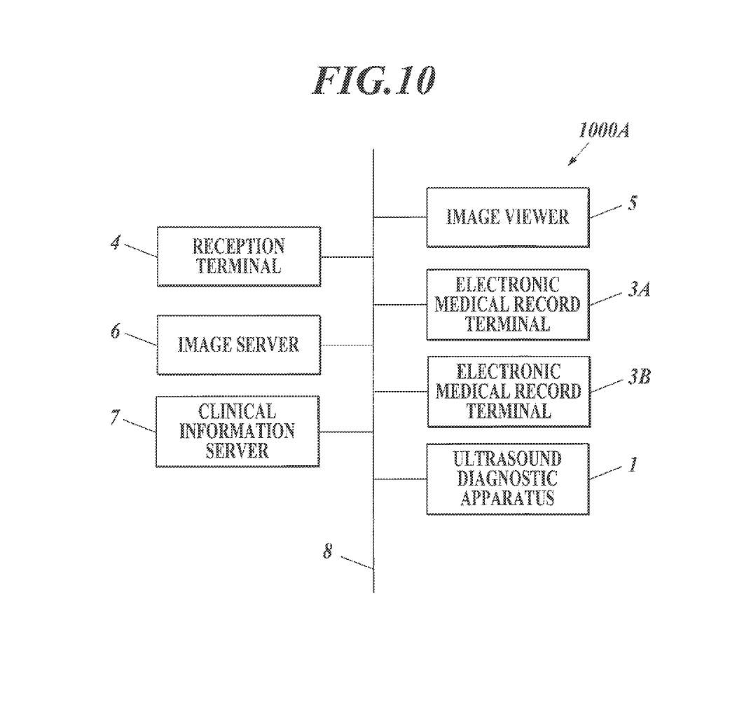

A second modification of the above-described embodiment will be described with reference to FIGS. 10 to 12. First, a configuration of the apparatus of the present modification will be described with reference to FIG. 10. FIG. 10 is a block diagram illustrating a medical inspection system 1000A. Note that, in the apparatus configuration of the present modification, the same reference numerals are given to the same portions as those of the medical inspection system 1000 according to the above-described embodiment, and the description thereof will be omitted.

As illustrated in FIG. 10, the medical inspection system 1000A includes the ultrasound diagnostic apparatus 1, an electronic medical record terminal 3A, an electronic medical record terminal 3B, the reception terminal 4, the image viewer 5, the image server 6, and the clinical information server 7. Each of the apparatuses and devices of the medical inspection system 1000A is connected with each other via the LAN 8.

Each of the electronic medical record terminals 3A and 3B has a configuration similar to the configuration of the electronic medical record terminal 3 of the above-described embodiment. Herein, the hospital as a medical facility in which the medical inspection system 1000A is installed has a first examination room and a second examination room. The electronic medical record terminal 3A is installed in the first examination room. The electronic medical record terminal 3B and the ultrasound diagnostic apparatus 1 are installed in the second examination room.

Moreover, the ROM of the controller 107 of the ultrasound diagnostic apparatus 1 stores a third inspection program instead of the first inspection program, and the storage 75 of the clinical information server 7 stores a second medical record management program.

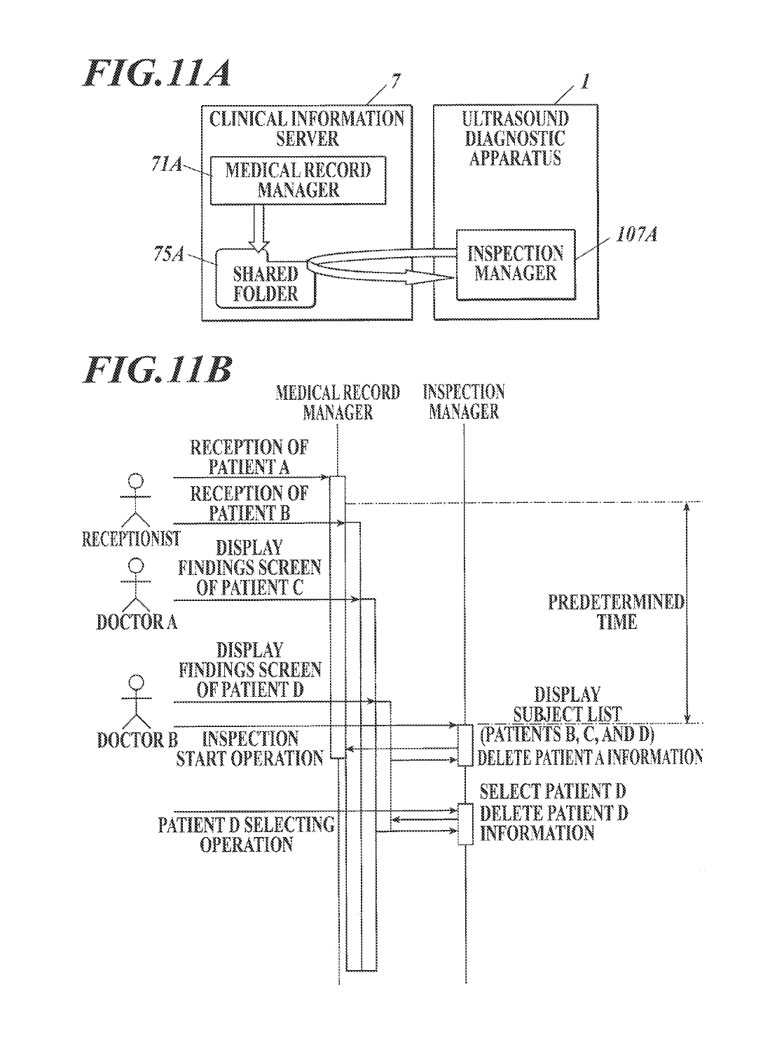

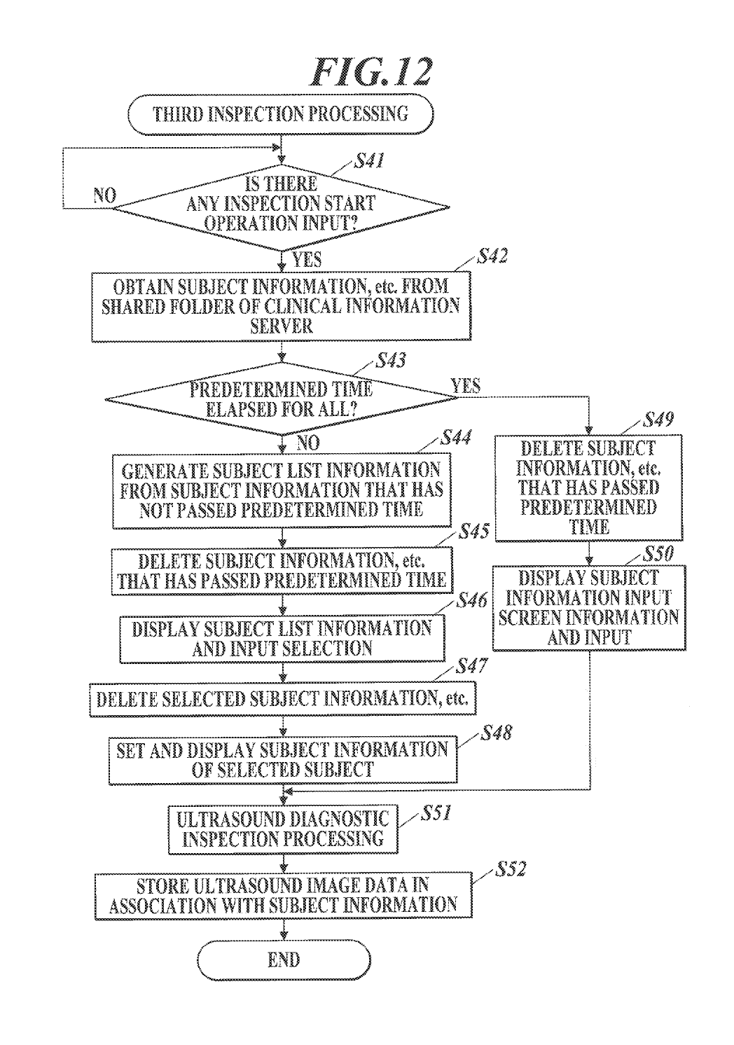

Next, operation of the medical inspection system 1000A will be described with reference to FIGS. 11A to 12. FIG. 11A is a block diagram illustrating a functional configuration of the clinical information server 7 and the ultrasound diagnostic apparatus 1 in third inspection processing. FIG. 11B is a sequence diagram illustrating a specific example of the operation related to the third inspection processing. FIG. 12 is a flowchart illustrating the third inspection processing.

First, as illustrated in FIG. 10A, in the clinical information server 7, the CPU 71 executes a second medical record management processing according to the second medical record management program stored in the storage 75. A medical record manager 71A is determined as an execution subject of the second medical record management processing. Moreover, the storage 75 stores subject information of an individual patient as a caption file of the electronic medical record, and also stores a shared folder 75A. Internal data of the shared folder 75A can be updated and deleted from the external ultrasound diagnostic apparatus 1.

Similarly to the electronic medical record terminal 3, each of the electronic medical record terminals 3A and 3B has the medical record manager 31A. The medical record manager 31A of the electronic medical record terminals 3A and 3B generates subject information and subject information update time information when displaying findings screen information including the electronic medical record. Then, the medical record manager 31A of the electronic medical record terminals 3A and 3B stores the generated subject information and subject information update time information in the shared folder 75A via the communicator 36. Moreover, at reception of a visiting patient, the reception terminal 4 generates subject information and subject information update time information and stores the generated information in the shared folder 75A. In this manner, the shared folder 75A stores the subject information and subject information update time information of the patient at the time of reception, and stores the subject information and the subject information update time information of the examination target patient in the first examination room and the second examination room.

Then, triggered by an input of the third inspection processing execution instruction by the doctor via the operation receiver 103 in the ultrasound diagnostic apparatus 1, for example, the controller 107 executes the third inspection processing in accordance with the third inspection program stored in the ROM. The inspection manager 107A is determined as the execution subject of the third inspection processing.

As illustrated in FIG. 12, Step S41 is similar to Step 511 of the first inspection processing in FIG. 6. In a case where the inspection start operation input is performed (Step S41; YES), the inspection manager 107A acquires all the subject information and the subject information update time information from the shared folder 75A of the clinical information server 7 via the communicator 109 (Step S42).

Then, the inspection manager 107A acquires the current date and time information from the time counter 110, and determines whether a predetermined time fixedly set in advance has elapsed on the basis of all the subject information update time information acquired in Step S42 using the acquired current date and time information (Step S43). The predetermined time in Step S43 is a passage of time for determining whether there is occurrence of examination finish from the reception in addition to whether the findings screen of the examined patient is erroneously left on, and an exemplary predetermined time is 20 minutes.

If the predetermined time has not elapsed (Step S43: NO), the inspection manager 107A generates subject list information, that is, a list of names of patients for which the predetermined time has not elapsed from the subject information that has not passed a predetermined time from the subject information update time information among all the subject information acquired in Step S42 (Step S44). Then, the inspection manager 107A deletes the subject information and the subject information update time information that has passed the predetermined time from the shared folder 75A via the communicator 109 (Step S45).

Then, the inspection manager 107A displays the subject list information generated in Step S44 on the display 106, and receives a selection input of the next examination target patient from among the subject list information by the doctor via the operation receiver 103 (Step S46). Then, the inspection manager 107A deletes the subject information and the subject information update time information selected in Step S46 from the shared folder 75A via the communicator 109 (Step S47). Then, the inspection manager 107A sets the subject information acquired in Step S42 corresponding to the patient selection information input in Step S46 as the subject information of the inspection target patient and displays the set information on the display 106 (Step S48).

In a case where the predetermined time has elapsed (Step S43; YES), the inspection manager 107A deletes the subject information and the subject information update time information that have passed a predetermined time from the shared folder 75A via the communicator 109 (Step S49). Steps S50 and S51 are similar to Steps S15 and S16 of the first inspection processing in FIG. 6. On the basis of the input of an instruction to store the ultrasound image data by the doctor via the operation receiver 103, the inspection manager 107A stores storage target ultrasound image data generated in Step S51 in the image server 6 (Step S52) in association with the subject information acquired in Step S46 or input in S49, via the communicator 109, and finishes the third inspection processing.

Next, a specific example of the operation related to the third inspection processing will be described with reference to FIG. 11B. Firstly, patients C and D who have finished reception are in the waiting room, subject information and subject information update information of the patients C and D at the time of reception are stored in the shared folder 75A of the clinical information server 7. Then, a visiting patient A finishes reception with the guidance of a receptionist and subject information and subject information update information of the patient A at the time of reception are stored in the shared folder 75A by the reception terminal 4. Then, a visiting patient B finishes reception and subject information and subject information update information of the patient B at the time of reception are stored in the shared folder 75A by the reception terminal 4.

Herein, a doctor A is arranged in the first examination room and a doctor B is arranged in the second examination room. Then, the patient C enters the first examination room with the guidance of the doctor A. Then, the doctor A performs operation input of an instruction to display a findings screen including the electronic medical record of the inspection target patient C via the operation receiver 32 of the electronic medical record terminal 3A, and then, the findings screen information is displayed on the display 34. In response to this, the medical record manager 31A of the electronic medical record terminal 3A updates and stores the subject information of the examination target patient C together with the subject information update time information at the time of display of the findings screen, in the shared folder 75A. Then, the patient D enters the second examination room with the guidance of the doctor B. Then, the doctor B performs operation input of an instruction to display a findings screen of the examination target patient D via the operation receiver 32 of the electronic medical record terminal 3B, and then, the findings screen information is displayed on the display 34. In response to this, the medical record manager 31A of the electronic medical record terminal 3B updates and stores the subject information of the examination target patient D together with the subject information update time information at the time of display of the findings screen, in the shared folder 75A.

Then, the doctor B performs operation input of an ultrasound diagnosis inspection start instruction for the inspection target patient via the operation receiver 103 of the ultrasound diagnostic apparatus 1, and the inspection manager 107A reads, in Step S42, the subject information and the subject information update time information of all the patients A, B, C, and D from the shared folder 75A. Then, in Step S43, the inspection manager 107A determines that the read subject information update time information of the patients B, C, and D have not passed a predetermined time from the current date and time information, generates, in Steps S44 and S45, the subject list information of the patient B, C, and D, and deletes, from the shared folder 75A, the subject information and the subject information update time information of the patient A, that has passed the predetermined time.

Then, in Steps S46 and S47, the inspection manager 107A displays the subject list information of the patients B, C, and D on the display 106, and receives, via the operation receiver 103, patient selection information of the patient D from the doctor B, and deletes the subject information and the subject information update time information of the selected patient D, from the shared folder 75A. In Step S48, the inspection manager 107A sets the selected subject information as the subject information of the inspection target patient. Then, in Steps S51 and S52, the inspection manager 107A starts ultrasound diagnostic inspection and stores the ultrasound image data generated in the inspection in the image server 6 in association with the subject information of the inspection target patient D.

As described above, according to the present modification, the clinical information server 7 stores the subject information of and the subject information update time information of the plurality of patients. The inspection manager 107A acquires the last updated subject information and subject information update time information of the plurality of patients from the clinical information server 7 at the timing of execution of the predetermined operation input, generates the subject list information indicating the subject of the subject information that is within a predetermined time from the subject information update time information among the plurality of acquired subject information and the subject information update time information, and displays the generated subject list information on the display 106. The operation receiver 103 receives a selection input of the subject based on the subject list information. The inspection manager 107A sets the subject information of the selected subject as the subject information of the inspection target subject. With this configuration, it is possible to easily acquire select, and accurately set the subject information of the inspection target patient.

### Third Modification

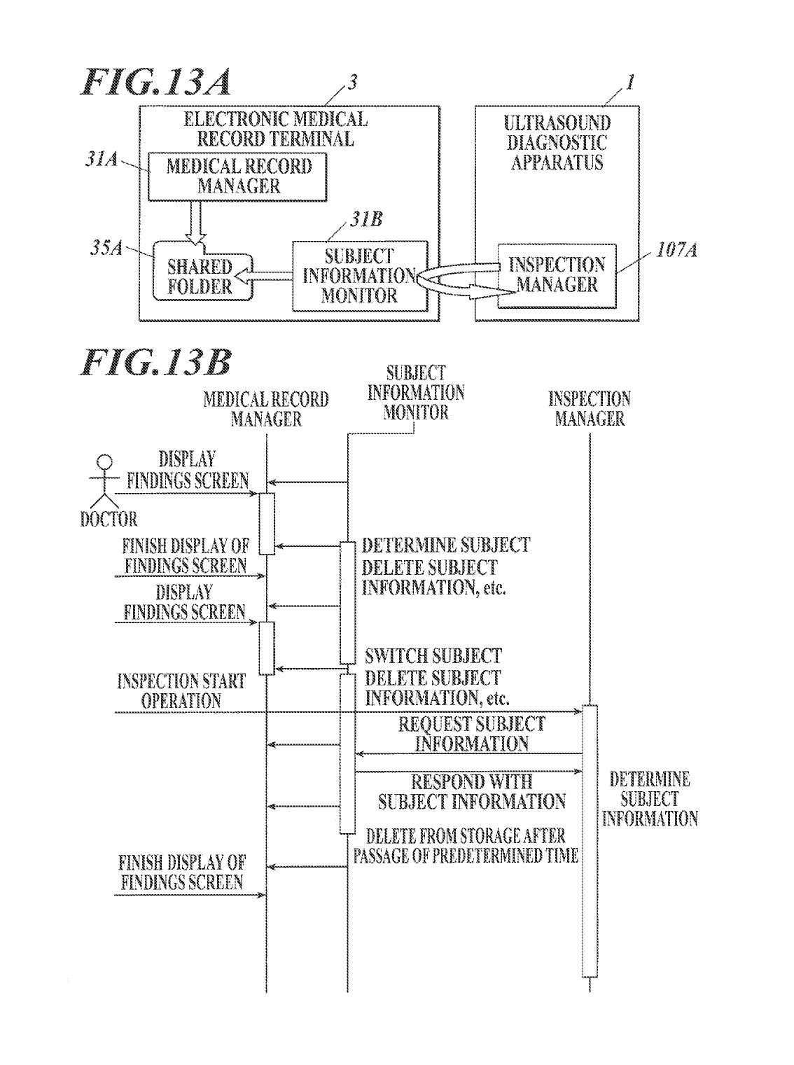

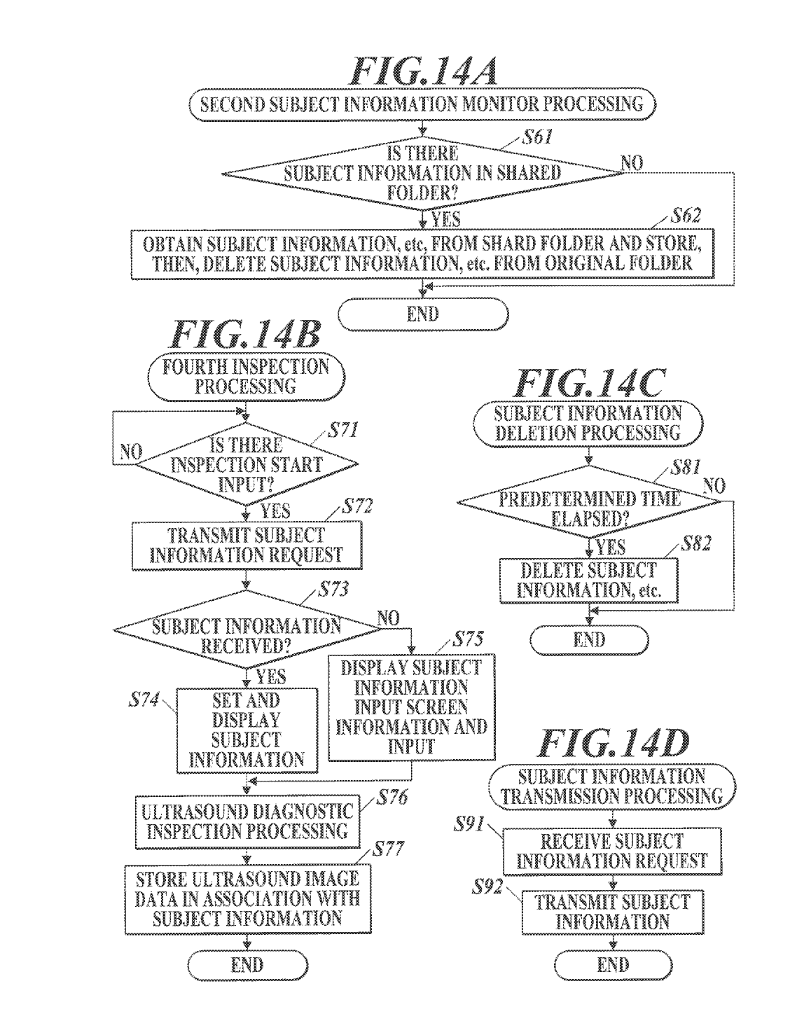

A third modification of the above-described embodiment will be described with reference to FIGS. 13A to 14D. Similarly to the above-described embodiment, the medical inspection system 1000 is used as the apparatus configuration of the present modification. Note that the ROM of the controller 107 of the ultrasound diagnostic apparatus 1 stores a fourth inspection program instead of the first inspection program. Herein, in addition to the first medical record management program, a second subject information management program, a subject information deletion program, and a subject information transmission program are stored in the storage 35 of the electronic medical record terminal 3.

Next, the operation of the medical inspection system 1000 will be described with reference to FIGS. 13A to 14D. FIG. 13A is a diagram illustrating a functional configuration of the electronic medical record terminal 3 and the ultrasound diagnostic apparatus 1 in second subject information monitor processing, fourth inspection processing, subject information deletion processing, and subject information transmission processing. FIG. 13B is a sequence diagram illustrating a specific example of operation related to the second subject information monitor processing, the fourth inspection processing, the subject information deletion processing, and the subject information transmission processing. FIG. 14A is a flowchart illustrating the second subject information monitor processing. FIG. 14B is a flowchart illustrating the fourth inspection processing. FIG. 14C is a flowchart illustrating the subject information deletion processing. FIG. 14D is a flowchart illustrating the subject information transmission processing.

First, as illustrated in FIG. 13A, similarly to the above-described embodiment, the medical record manager 31A is determined as the execution subject of the medical record management processing in the electronic medical record terminal 3, and accordingly, the medical record manager 31A stores electronic medical records in the electronic folders, generates subject information and subject information update time information of the examination target patient and stores the generated information in the shared folder 35A in accordance with the operation input of the instruction to display the findings screen of the next examination target patient.

In parallel with this, the second subject information monitor processing is executed. The second subject information monitor processing is executed repeatedly at a predetermined cycle. In the electronic medical record terminal 3, triggered by the passage of a predetermined cycle of the current date and time information of the time counter 37 from the previous execution of the second subject information monitor processing, the CPU 31 executes the second subject information monitor processing in accordance with a second subject information monitor program stored in the storage 35. A subject information monitor 31B is determined as the execution subject of the second subject information monitor processing.

First, as illustrated in FIG. 14A, the subject information monitor 31B checks data in the shared folder 35A and determines whether the subject information is stored (Step S61). In a case where the subject information is not stored (Step S61; NO), the second subject information monitor processing is finished. In a case where the subject information is stored (Step S61; YES), the subject information monitor 31B acquires subject information and subject information update time information of the next examination target patient from the shared folder 35A and stores the acquired information in a location (for example, a predetermined folder (not illustrated)) other than the shared folder 35A of the storage 35, and together with this, deletes the acquired subject information and the subject information update time information from the shared folder 35A (Step S62), and finishes the second subject information monitor processing.

Then, triggered by an input of the third inspection processing execution instruction by the doctor via the operation receiver 103 in the ultrasound diagnostic apparatus 1, for example, the controller 107 executes the fourth inspection processing in accordance with the fourth inspection program stored in the ROM. The inspection manager 107A is determined as the execution subject of the fourth inspection processing.

As illustrated in FIG. 14B, Step S71 is similar to Step S11 of the first inspection processing in FIG. 6. In a case where the inspection start operation input is performed (Step S71; YES), the inspection manager 107A transmits a subject information request to the electronic medical record terminal 3 (subject information monitor 31B) via the communicator 109 (Step S72). Then, the inspection manager 107A determines whether the subject information has been received from the electronic medical record terminal 3 (subject information monitor 31B) via the communicator 109 (Step S73).

In a case where the subject information has been received (Step S73; YES), the inspection manager 107A sets the subject information received in Step S73 as the subject information of the inspection target patient and displays the set information on the display 106 (Step S74). In a case where subject information has not been received due to timeout (Step S73; NO), the processing proceeds to Step S75. Steps S75 and S76 are similar to Steps S15 and S16 of the first inspection processing in FIG. 6. On the basis of the input of an instruction to store the ultrasound image data from the doctor via the operation receiver 103, the inspection manager 107A stores storage target ultrasound image data generated in Step S76 in the image server 6 (Step S77) in association with the subject information acquired in Step S73 or input in S75, via the communicator 109, and finishes the fourth inspection processing.

Moreover, in parallel with the second subject information monitor processing and the fourth inspection processing, the subject information deletion processing is executed. The subject information deletion processing is executed repeatedly at a predetermined cycle. In the electronic medical record terminal 3, triggered by the passage of a predetermined cycle of the current date and time information of the time counter 37 from the execution of the subject information deletion processing, the CPU 31 executes the subject information deletion processing in accordance with the subject information deletion program stored in the storage 35. The subject information monitor 31B is determined as the execution subject of the subject information deletion processing.

As illustrated in FIG. 14C, the subject information monitor 31B reads the subject information and the subject information update time information from a location other than the shared folder 35A of the storage 35, acquires the current date and time information from the time counter 37, and determines, using the acquired current date and time information, whether a predetermined time fixedly set in advance has elapsed from the read subject information update time information (Step S81). The predetermined time in Step S81 is a passage of time for determining whether the findings screen of the examined patient is erroneously left on, and an exemplary predetermined time is five minutes.

In a case where the predetermined time has elapsed (Step S81; YES), the subject information monitor 31B deletes the subject information and the subject information update time information read in Step S82 from the storage 35 (Step S82), and finishes the subject information deletion processing. In a case where the predetermined time has not elapsed (Step S81; NO), the subject information deletion processing is finished.

Moreover, in parallel with the fourth inspection processing, the subject information transmission processing is executed. In the electronic medical record terminal 3, in response to Step S72 in the fourth inspection processing, triggered by the start of reception of the subject information request from the ultrasound diagnostic apparatus 1 (inspection manager 107A) via the communicator 36, the CPU 31 executes the subject information transmission processing in accordance with the subject information transmission program stored in the storage 35. The subject information monitor 31B is determined as the execution subject of the subject information transmission processing.

As illustrated in FIG. 14D, in response to Step S72, the subject information monitor 31B completes reception of the subject information request from the ultrasound diagnostic apparatus 1 (inspection manager 107A) via the communicator 36 (Step S91). Then, the subject information monitor 31B reads the subject information from the storage 35 in response to Step S73, and transmits the read subject information to the ultrasound diagnostic apparatus 1 (inspection manager 107A) via the communicator 36 (Step S92), and finishes the subject information transmission processing.

Next, a specific example of operation related to the second subject information monitor processing, the fourth inspection processing, the subject information deletion processing, and the subject information transmission processing will be described with reference to FIG. 13B. First, a first examination target patient who has finished reception enters the examination room. Subsequently, the doctor performs operation input of an instruction to display the findings screen including the electronic medical record of the examination target patient via the operation receiver 32, and then, the findings screen information is displayed on the display 34. Subsequently, the medical record manager 31A of the electronic medical record terminal 3 stores the subject information of the first examination target patient together with the subject information update time information, in the shared folder 35A.

In parallel with this, the subject information monitor 31B executes the second subject information monitor processing repeatedly at a predetermined cycle. Subsequently, in Step S62, the subject information monitor 31B acquires the subject information and the subject information update time information of the first examination target patient from the shared folder 35A, stores the acquired information in the storage 35, and deletes the acquired subject information and the subject information update time information in the shared folder 35A.

The first examination target patient goes through the examination without undergoing inspection using the ultrasound diagnostic apparatus 1 and leaves the examination room. After the examination is finished, the doctor performs operation input of an instruction to finish the display of the findings screen for the first examination target patient via the operation receiver 32, and then, the display of the findings screen information on the display 34 is finished. Subsequently, a second examination target patient who has finished reception next enters the examination room. Then, the doctor performs operation input of an instruction to display the findings screen including the electronic medical record of the second examination target patient via the operation receiver 32, and the findings screen information is displayed on the display 34. In response to this, the medical record manager 31A stores the subject information of the second examination target patient together with the subject information update time information, in the shared folder 35A.

Subsequently, in Step S62, the subject information monitor 31B acquires the subject information and the subject information update time information of the second examination target patient from the shared folder 35A, stores the acquired information in the storage 35 (while the subject information and the subject information update time information for the first patient is deleted), and deletes the acquired subject information and the subject information update time information in the shared folder 35A.

Then, the doctor performs operation input of an ultrasound diagnosis inspection start instruction for the second inspection target patient via the operation receiver 103 of the ultrasound diagnostic apparatus 1, and the inspection manager 107A transmits, in Step S72, the subject information request to the subject information monitor 31B, and then, the inspection manager 107A receives, in Step S73, the subject information of the second patient from the subject information monitor 31B. In Step S74, the inspection manager 107A sets the received subject information as the subject information of the inspection target patient.

Then, in Steps S76 and S77, the inspection manager 107A starts ultrasound diagnostic inspection and stores the ultrasound image data generated in the inspection in the image server 6 in association with the subject information of the inspection target patient. In parallel with this, the subject information monitor 31B executes the subject information deletion processing repeatedly at a predetermined cycle. Subsequently, when the subject information update time information stored in the storage 35 has passed a predetermined time in Step S81, the subject information monitor 31B deletes in Step S82 the subject information of the second examination target patient from the storage 35. After the examination is finished, the doctor performs operation input of an instruction to finish the display of the findings screen of the second examination target patient via the operation receiver 32, and then, the display of the findings screen information on the display 34 is finished.

As described above, according to the present modification, the electronic medical record terminal 3 includes the storage 35 that stores subject information and subject information update time information of the patient who comes to a hospital, and includes the CPU 31 (subject information monitor 31B) that deletes the subject information and the subject information update time information that have passed a predetermined time from the subject information update time information from among the stored subject information and the subject information update time information, and responds by reading the last updated subject information from the storage 35 in response to the subject information request transmitted from the ultrasound diagnostic apparatus 1. The ultrasound diagnostic apparatus 1 includes the inspection manager 107A that requests the last updated subject information to the electronic medical record terminal 3 at the timing of execution of a predetermined operation input, acquires responded subject information from the electronic medical record terminal 3, and sets the acquired subject information as the subject information of the inspection target patient. With this configuration, it is possible to easily acquire the subject information of the inspection target patient using the electronic medical record terminal 3 having a function to respond to the subject information.

Note that the description in the above-described embodiment is an example of a preferred medical inspection apparatus and a medical inspection system according to the present invention, which is not limited to this. For example, at least two of the configurations of the above-described embodiment and each of the modifications may be appropriately combined with each other.

In the above-described embodiment and modifications, the ultrasound diagnostic apparatus 1 is used as the medical inspection apparatus, but the present invention is not limited to this. The medical inspection apparatus may be represented by another inspection apparatus such as an electrocardiogram inspection apparatus, an X-ray radiography apparatus (computed radiography (CR) apparatus).

In the above-described embodiment and modifications, the ultrasound image data as the inspection data generated by the ultrasound diagnostic apparatus 1 is stored in association with the subject information, but the present invention is not limited to this. The inspection data are data produced as a result of inspection and includes image data generated by inspection and measurement data such as numerical values as measurement results. It is allowable to be a configuration of storing other inspection data including the measurement data other than the ultrasound image data generated by the ultrasound diagnostic apparatus 1, measurement data of the electrocardiogram generated by the electrocardiogram as an inspection target apparatus, X-ray image data captured by the X-ray imaging apparatus, or the like, in association with the subject information.

Furthermore, in the above-described embodiment and modifications, the ultrasound image data as the inspection data generated by the ultrasound diagnostic apparatus 1 are output and stored in association with the subject information, to the image server 6 as the output destination. It is, however, not limited to this configuration. It is allowable to have a configuration in which subject information is associated with inspection data generated by a medical inspection apparatus and is output to and stored in the storage 108 as an output destination, and a configuration in which output and storage are performed to (a storage of) an external device as an output destination other than the image server 6, a configuration for outputting and displaying the information on a display in the medical inspection apparatus or on a display of an external device as an output destination, and a configuration for outputting and printing the information to/in an external printing device.

Moreover, while in the above-described embodiment and modifications, each of the predetermined time used for time passage determination of the subject information update time information is fixedly set in advance, the configuration is not limited to this. It is allowable to have a configuration in which setting of the individual predetermined times can be changed appropriately in accordance with an operation input from a user (doctor, or the like,) via the operation receiver 103 and the operation receiver 32. Moreover, each of the predetermined times used for time passage determination of the subject information update time information may be set to an infinitely long time.

Furthermore, each of the predetermined times used for time passage determination of the subject information update time information may be variable (to be changed automatically). For example, when the medical facility is crowded with patients, each of the predetermined times is set to be longer, and when it is vacant, each of the predetermined times is set shorter. Whether it is crowded or not may be determined by the update frequency of the caption file. In a case where the caption file is frequently updated, each of the predetermined times is increased since the waiting time is considered to be long, and in the case where the caption file is not frequently updated, each of the predetermined times is decreased. Alternatively, it is also allowable to configure such that, in a case where the caption file is frequently updated, each predetermined time is decreased since the waiting time is considered to be short, and in the case where the caption file is not frequently updated, each of the predetermined times is increased.

Moreover, while in the above-described embodiment and modifications, the subject information input screen information is displayed and the operation input of the subject information is received in a case where the subject information update time information has passed a predetermined time, the configuration is not limited to this. It is also allowable to configure such that, in the ultrasound diagnostic apparatus 1, the inspection manager 107A displays an alert screen information including the subject information and a report that the subject information update time information has passed the predetermined time, on the display 106 in a case where the subject information update time information has passed the predetermined time. Furthermore, it is also allowable to configure such that the inspection manager 107A displays the alert screen information on the display 106 and displays the subject information input screen information and receives an operation input of the subject information via the operation receiver 103.

Moreover, while in the above-described embodiment and modifications, the operation input of inspection start is set as a trigger for acquisition of the subject information by the inspection manager 107A in the ultrasound diagnostic apparatus 1, the configuration is not limited to this. For example, it is also allowable to set operation other than the above, such as storage (save) instruction of generated ultrasound image data, as the trigger for acquisition of the subject information.

Moreover, while in the above-described embodiment and modifications, the subject information update time information uses the timing of instructing the display of the findings screen information or the timing of generating the subject information at the reception of examination, the timing is not limited to this. For example, it is allowable to configure such that the time information of another timing, such as at registration of subject information of a new patient, at subject information change, and at order issuance, is used as the subject information update time information.

Moreover, it is possible to appropriately add modification to the details in the structure and operation of individual portions constituting the medical inspection systems 1000 and 1000A in the above-described embodiment within the scope and spirit of the present invention.

Although embodiments of the present invention have been described and illustrated in detail, it is clearly understood that the same is by way of illustration and example only and not limitation, the scope of the present invention should be interpreted by terms of the appended claims.

What is claimed is:

1. A medical inspection apparatus which is capable of communicating with an external device storing at least one piece of subject information of a subject and at least one piece of subject information update time information indicating an update time of the subject information and which inspects the subject to generate inspection data, the medical inspection apparatus comprising:
an acquisitor which acquires the last updated subject information and the subject information update time information corresponding to the last updated subject information at a timing of execution of a predetermined operation input; and
a setter which sets the acquired subject information as the subject information of an inspection target subject when it is within a predetermined time from the acquired subject information update time information.

2. The medical inspection apparatus of claim 1, wherein the predetermined operation input is an operation input of starting inspection or storing the inspection data.

3. The medical inspection apparatus of claim 1, wherein the acquisitor acquires the last updated subject information and the subject information update time information corresponding to the last updated subject information from the external device at the timing of execution of the predetermined operation input.

4. The medical inspection apparatus of claim 1, further comprising:
a storage; and
a monitor which checks whether the external device stores the subject information at a predetermined cycle, and which acquires the subject information and the subject information update time information corresponding to the subject information to store the acquired subject information and the acquired subject information update time information in the storage when the external device stores the subject information,
wherein the acquisitor reads and acquires the last updated subject information and the subject information update time information corresponding to the last updated subject information from the storage at the timing of execution of the predetermined operation input.

5. The medical inspection apparatus of claim 1, wherein
the external device stores a plurality of pieces of the subject information and a plurality of pieces of the subject information update time information of a plurality of subjects,
the acquisitor acquires the plurality of pieces of the subject information and the plurality of pieces of the subject information update time information from the external device at the timing of execution of the predetermined operation input,
the medical inspection apparatus further comprises:
a display controller which generates subject list information indicating the subject of the subject information which is within the predetermined time from the subject information update time information corresponding to the subject information, from among the acquired plurality of pieces of the subject information and the acquired plurality of pieces of the subject information update time information, and which displays the generated subject list information on a display; and
a selector which receives a selection input of the subject based on the subject list information, and
the setter sets the subject information of the selected subject as the subject information of the inspection target subject.

6. The medical inspection apparatus of claim 1, further comprising:
an inspector which inspects the subject to generate the inspection data; and
an output controller which outputs the generated inspection data in association with the set subject information, to an output destination.

7. The medical inspection apparatus of claim 1, further comprising an operation receiver which receives an operation input of the subject information of the inspection target subject when the predetermined time has elapsed from the acquired subject information update time information.

8. A medical inspection system comprising an external device and a medical inspection apparatus which is capable of communicating with the external device and inspects a subject to generate inspection data,
wherein the external device includes:
a storage which stores subject information of the subject coming to a medical facility and stores subject information update time information indicating an update time of the subject information;
a deleter which deletes the subject information having passed a predetermined time from the subject information update time information corresponding to the subject information and deletes the subject information update time information, from among the stored subject information and the stored subject information update time information; and
a responsor which responds to a request for the subject information from the medical inspection apparatus by reading the last updated subject information from the storage, and
the medical inspection apparatus includes:
an acquisitor which requests the last updated subject information to the external device at a timing of execution of a predetermined operation input and acquires the responded subject information from the external device; and
a setter which sets the acquired subject information as the subject information of an inspection target subject.
